(19) United States
(12) Patent Application Publication
(10) Pub. No.: US 2025/0326757 A1
(43) Pub. Date: Oct. 23, 2025

(54) SOLID FORMS OF HETEROCYCLYLAMIDES AS IRAK4 INHIBITORS

(71) Applicant: AstraZeneca AB, Södertälje (SE)

(72) Inventors: Okky PUTRA, Södertälje (SE); Ina TERSTIEGE, Södertälje (SE); Anna Ingrid Kristina BERGGREN, Södertälje (SE)

(21) Appl. No.: 18/870,598

(22) PCT Filed: May 25, 2023
(86) PCT No.: PCT/EP2023/064007

§ 371 Date: Nov. 29, 2024

Related U.S. Application Data
(60) Provisional application No. 63/365,387, filed on May 26, 2022.

(51) Int. Cl. C07D 487/04; C07D 231/56

(52) U.S. Cl. CPC C07D 487/04; C07D 231/56

(57) ABSTRACT

The present specification relates to novel physical forms of a indazole-5-carboxamide derivative, as well as solvate and salt forms of the same compound. A process for the preparation of the compound and uses of the new physical forms are also provided.

The specification relates to polymorph, salts, co-crystal and solvate forms of N-(Imidazo[1,2-b]pyridazin-3-yl)-6-methoxy-2-((1r,4r)-4-(N-methylacetamido)cyclohexyl)-2H-indazole-5-carboxamide (Compound (I)), to pharmaceutical compositions containing them and their use in therapy. The specification also relates to a chemical process for the production of Compound (I). Compound (I) has been discovered to be a highly active inhibitor of IRAK4 and consequently has potential utility as a medicine for the treatment of respiratory diseases such as asthma and chronic obstructive pulmonary disease (COPD), of cancer, of inflammatory diseases and of autoinflammatory/autoimmune diseases such as systemic lupus erythematosus, rheumatoid arthritis, myositis, Sjögren's syndrome, systemic sclerosis, gout, endometriosis, atopic dermatitis and psoriasis.

Interleukin-1 receptor (IL-1R)-associated kinase 4 (IRAK4) is a key regulator of immune signaling. IRAK4 is expressed by multiple cell types and mediates signal transduction from Toll-like receptors (TLRs) and receptors of the interleukin-1 (IL-1) family, including IL-1R, IL-18R and the IL-33 receptor ST2. TLRs recognize and respond to ligands derived from microbes, such as lipopolysaccharide (LPS) or microbial RNA or DNA, while receptors of the IL-1 family can be activated by endogenous ligands produced by TLR-activated cells (IL-1B and IL-18) or by tissue damage (IL-1a and IL-33). Upon activation of TLRs or IL-1 receptors by their ligands, the adaptor protein myeloid differentiation primary response 88 (MyD88) is recruited to the receptor and forms a multimeric protein complex, called the “Myddosome”, together with proteins of the IRAK family (IRAK1, IRAK2 and IRAK4). The Myddosome serves as a signaling platform to induce nuclear factor κB (NF-κB) and mitogen-activated protein kinase (MAPK) signal transduction pathways, culminating in the activation of transcription factors NF-κB, activator protein 1 (AP1), c-AMP response element-binding protein (CREB) and interferon regulatory factor 5 (IRF5), driving transcription of inflammatory cytokines and chemokines. Mice lacking IRAK4 are viable but lack inflammatory cytokine response to IL-1β, IL-18 and LPS. Humans presenting loss-of-function mutations in IRAK4 display an immunocompromised phenotype and their immune cells show an abrogated cytokine response to TLR agonists and IL-1 receptor ligands.

IRAK4 is characterized by an N-terminal death domain that mediates the interaction with MyD88 and a centrally located kinase domain. Myddosome formation promotes IRAK4 auto-phosphorylation which modulates the stability and downstream signaling of the Myddosome. The kinase activity of IRAK4 is required for cytokine induction by TLRs and IL-1R, as shown by studies in knock-in mice expressing a kinase-dead IRAK4, as well as in studies using small molecule IRAK4 kinase inhibitors.

Given its critical role in eliciting an inflammatory response, IRAK4 constitutes a target for drugs that exert an anti-inflammatory effect.

Asthma and COPD (chronic obstructive pulmonary disease) are chronic lung diseases constituting a major unmet medical need around the world. Asthma and COPD are characterized by chronic airway inflammation, involving abnormal cytokine release, dysregulated immune cell activation and airway remodeling. In asthma, insults to the airways such as allergenic, viral and bacterial insults activate the TLR receptors via pathogen associated molecular patterns (PAMPs), and the IL-1R and ST2 receptors via the release of alarmins, including IL-33 and IL-1a, as well as by IL-1B released upon inflammasome activation. TLRs and receptors of the IL-1 family are present in multiple cell types in the airways, including macrophages, dendritic cells, mast cells, monocytes and epithelial cells, and respond to their ligands by releasing inflammatory cytokines (TNF-α, IL-6, IL-8, GM-CSF, IL-5) leading to airway inflammation, recruitment of inflammatory cells such as neutrophils and eosinophils, airway hyperresponsiveness and mucus production. IRAK4 inhibition has the potential to suppress these inflammatory pathways in the airways. Gene expression analysis of lung samples from asthma and COPD patients, have revealed an upregulated expression of genes associated with the IL-1R and TLR2/4 inflammatory pathways in subsets of severe patients. Although IRAK4 inhibitors have not, to the best of our knowledge, been explored in the clinic for the treatment of respiratory diseases, pre-clinical data from several research groups indicates that interfering with IRAK4-regulated pathways attenuates airway inflammation in animal models of both asthma and COPD. For instance, mice lacking MyD88, the central component of the myddosome, are protected against airway inflammation induced by allergens or IL-33, as are mice treated with a small molecule mimetics blocking the interaction between IRAK2 and IRAK4. Blocking IL-1B with a monoclonal antibody has also been found to suppress airway inflammation induced by allergens and bacteria in a steroid-resistant mouse model of asthma. Moreover, the treatment of mice with the IL-1R antagonist anakinra at the time of allergen challenge ameliorates asthma-like symptoms in a mouse model of allergic asthma. Chronic exposure to cigarette smoke is a major contributing factor to the development of COPD. In mice exposed to cigarette smoke, IL-1 signaling is central in mediating neutrophilic airway inflammation, and blocking IL-1 signaling with antibodies against IL-1α, IL-1β or the IL-1R can ameliorate the neutrophilic inflammation in the lung and reduce bacteria- or virus-induced exacerbations in cigarette smoke-exposed mice. Taken together, IRAK4 inhibition has potential to provide a broad anti-inflammatory effect in inflammatory respiratory diseases by simultaneously blocking several disease-relevant signaling pathways.

As a central regulator of the Myddosome, IRAK4 is also a promising therapeutic target in other inflammatory diseases driven by IL-1R-, TLR- or ST2-mediated mechanisms. As previously disclosed, IRAK4 plays a role in autoimmune disorders such as rheumatoid arthritis and systemic lupus erythematosus (SLE) (see e.g. WO2017207386 & WO2015150995). In SLE, immunocomplexes composed by autoantibodies and self-antigens, can drive TLR-dependent pathological signaling. In SLE pathogenesis, IRAK4 inhibition reportedly blocks the release of type I interferons and pro-inflammatory cytokines mediated by TLR7 and TLR9 activation in plasmacytoid dendritic cells. Mice expressing a kinase-dead mutant of IRAK4 or treated with IRAK4 kinase inhibitor compounds, are resistant to experimentally induced arthritis and lupus (see e.g. WO2017207386). The approved use of anakinra (an IL-1 receptor antagonist) for the treatment of rheumatoid arthritis, also support the role of pathogenic IL-1R signaling in this disease. In Sjögren's syndrome, TLRs are upregulated in PBMCs (peripheral blood mononuclear cells) and salivary glands and TLR activation can stimulate release of interferon and other inflammatory cytokines, suggested to be implicated in Sjögren's pathogenesis. MyD88 knockout mice also display reduced disease manifestations in an experimental mouse model of Sjögren's syndrome. Systemic sclerosis is a severe autoimmune disorder where IL-1R, TLR4, TLR8 and ST2-signaling can drive pathogenic mechanisms, including microvascular damage and fibrosis. Inhibition of IRAK4 as a treatment in systemic sclerosis would thus block multiple disease-relevant pathways simultaneously. In myositis, elevated levels of IL-1α and IL-1β can contribute to muscle tissue inflammation. Myositis patients have also been characterized with high type I interferon gene signature, that may be partly driven by TLR7/9 activation, and the relevance of IL-1R signaling was supported by an improved clinical outcome in myositis patients treated with anakinra in a smaller mechanistic clinical trial. As a central regulator of the IL-1R pathway, IRAK4 is also a promising target in the treatment of gout. Monosodium urate crystals, characteristically formed in gout sufferers, can trigger the activation of the inflammasome and release of IL-1β. The use of both canakinumab, an anti IL-1β monoclonal antibody or anakinra has demonstrated clinical efficacy in the treatment of gout flares. Elevated levels of IL-1β and IL-33 have also been found in patients with endometriosis. The importance of IRAK4 in the disease process of endometriosis was shown in a mouse model where oral administration of an IRAK4 inhibitor suppressed lesion formation. MyD88 knockout mice were also protected against the development of endometriosis in the same mouse model. IL-33/ST2 signaling is a key mechanism in atopic dermatitis, involved in the regulation of skin inflammation, epithelial barrier integrity and eosinophil recruitment. IL-33 can trigger eczema and dermatitis in mice in a MyD88-dependent manner. As a regulator of ST2 signaling and a central component of the myddosome, IRAK4 inhibition has the potential to inhibit pathogenic IL-33/ST2 signaling in atopic dermatitis. Both TLR7 and IL-1R mediated mechanisms have been suggested to be involved in psoriasis. Imiquimod (TLR/8 agonist) can induce psoriasis-like disease in mice in a MyD88-dependent manner. IL-1β is upregulated in psoriatic skin lesions and the IL-1β/IL-1R axis has been suggested to contribute to skin inflammation and regulate the production of IL-17, a critical cytokine released from TH17 cells in psoriasis pathogenesis. IRAK4 kinase activity has further been shown to be required for the regulation of TH17 differentiation and TH17-mediated diseases in vivo.

A number of IRAK4 kinase inhibitors are known and have been developed principally for use in oncology or inflammatory disease (see e.g. WO2015150995, WO2017207386, WO2017009806, WO2016174183, WO2018234342). A number of clinical trial exploring the therapeutic utility of IRAK4 inhibitors are in progress.

N-(Imidazo[1,2-b]pyridazin-3-yl)-6-methoxy-2-((1r,4r)-4-(N-methylacetamido)cyclohexyl)-2H-indazole-5-carboxamide is disclosed in PCT/EP2021/084916 alongside its activity as an inhibitor of IRAK4 enzyme (IC₅₀ 0.2 nM) and IRAK4 activity in Karpas-299 cells (IC₅₀ 5 nM). In order to study the therapeutic potential of this compound it is desirable to have solid forms of the compound with suitable properties for pharmaceutical development. This application describes novel polymorph, salt and solvate forms of N-(Imidazo[1,2-b]pyridazin-3-yl)-6-methoxy-2-((1r,4r)-4-(N-methylacetamido)cyclohexyl)-2H-indazole-5-carboxamide. The compound is structurally distinct from previously known IRAK4 inhibitors.

In the formulation of drug substances, it is important for the drug substance (active compound) to be in a form in which it can be conveniently handled and processed. This is of importance, not only from the point of view of obtaining a commercially-viable manufacturing process for the drug substance itself, but also from the point of view of subsequent manufacture of pharmaceutical formulations comprising the active compound and suitable excipients. The chemical stability and the physical stability of the active compound are important factors in determining the suitability of a solid form for use in the development of pharmaceutical formulations. The active compound, and formulations containing it, should be capable of being effectively stored over appreciable periods of time, without exhibiting any significant change in the physico-chemical characteristics (e.g. chemical composition, density, hygroscopicity and solubility) of the active compound. It is an object of the present specification to provide solid forms of N-(Imidazo[1,2-b]pyridazin-3-yl)-6-methoxy-2-((1r,4r)-4-(N-methylacetamido)cyclohexyl)-2H-indazole-5-carboxamide suitable for pharmaceutical development. In a further object, the specification provides an expeditious route for the production of N-(Imidazo[1,2-b]pyridazin-3-yl)-6-methoxy-2-((1r,4r)-4-(N-methylacetamido)cyclohexyl)-2H-indazole-5-carboxamide.

This application relates to crystalline forms of N-(Imidazo[1,2-b]pyridazin-3-yl)-6-methoxy-2-((1r,4r)-4-(N-methylacetamido)cyclohexyl)-2H-indazole-5-carboxamide (hereafter “Compound (I)”). The structure of Compound (I) is shown below:

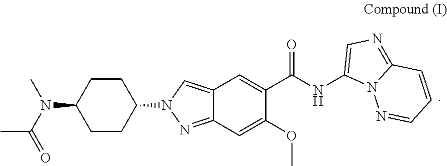 [SMILES: COc1cc2nn([C@H]3CC[C@H](N(C)C(C)=O)CC3)cc2cc1C(=O)Nc1cnc2cccnn12]

The application also relates to a process for producing Compound (I) as well as key synthetic intermediates.

We have found that Compound (I) may exist in a number of crystalline and salt forms.

One aspect provides a crystalline form of Compound (I).

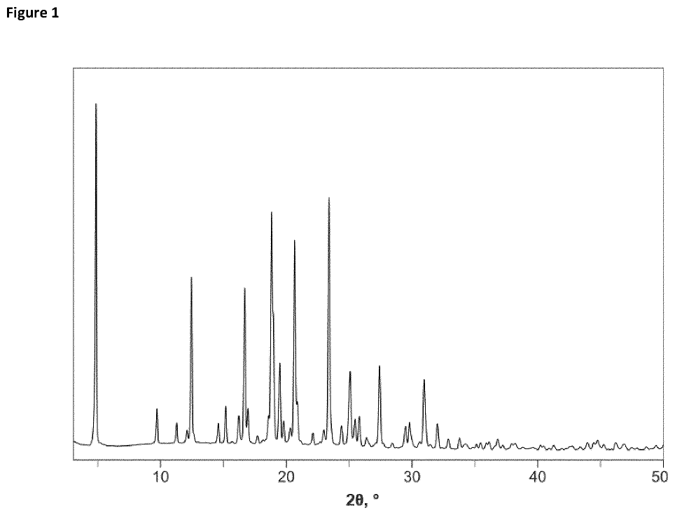

In a further aspect there is provided a physical form of Compound (I), Form A. “Form A”, provides an X-ray diffraction pattern substantially as shown in FIG. 1. Form A is an anhydrous crystalline form of Compound (I). Form A is the most thermodynamically stable anhydrous crystalline form of Compound A identified to date and is stable to prolonged storage under accelerated ageing conditions (40° C. and 75% relative humidity).

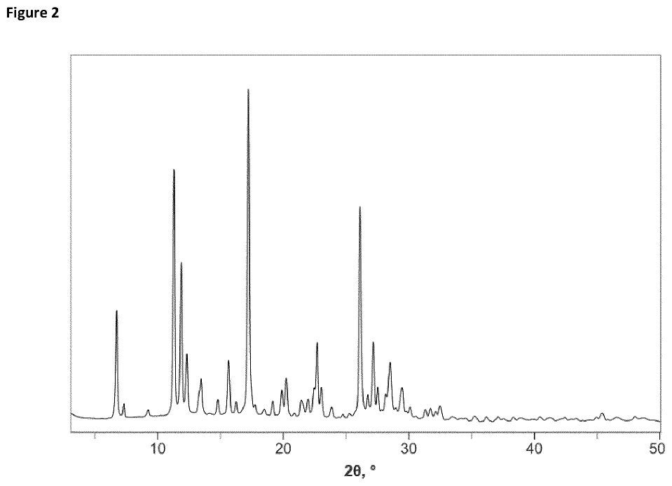

In a further aspect there is provided a physical form of Compound (I) Form B. “Form B” provides an X-ray diffraction pattern substantially as shown in FIG. 2. Form B is a trihydrate crystalline form of Compound (I).

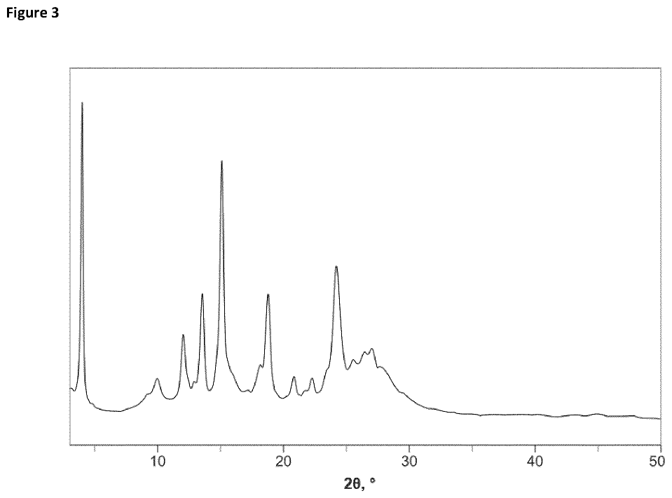

There is also described a meta stable dihydrate crystalline form of Compound (I). “Form C” provides an X-ray diffraction pattern substantially as shown in FIG. 3. Form B is an dihydrate crystalline form of Compound (I).

In a further aspect there is provided a crystalline solvate form of Compound (I).

In a further aspect there is provided a crystalline hydrate form of Compound (I), for example a trihydrate form.

In a further aspect there is provided a crystalline oxalate salt form of Compound (I).

In a further aspect there is provided a crystalline form comprising Compound (I) and 3-hydroxybenzoic acid, herein referred to as Compound (I) 3-hydroxybenzoic acid form or Compound (I) 3-hydroxybenzoic acid.

In a further aspect there is provided a crystalline form of Compound (I) for use in the manufacture of a medicament.

In a further aspect there is provided a crystalline form of Compound (I) for use in the manufacture of a medicament for use in the prevention or treatment of respiratory diseases such as asthma and chronic obstructive pulmonary disease (COPD).

In a further aspect there is provided a crystalline form of Compound (I) for use in the manufacture of a medicament for use in the prevention or treatment of cancer, for example a haematologic malignancy selected from Waldenstrom's macroglobulinemia (WM), non-Hodgkin lymphoma (NHL), diffuse large B-cell lymphoma (DLBCL), primary central nervous system lymphoma (PCNSL), Splenic Marginal Zone Lymphoma (SMZL), small lymphocytic lymphoma (SLL), leukaemias (chronic lymphocytic leukaemia (CLL)) and monoclonal gammopathy of undetermined significance (MGUS-IgM+).

In a further aspect there is provided a crystalline form of Compound (I) for use in the manufacture of a medicament for use in the prevention or treatment of inflammatory diseases and of autoinflammatory/autoimmune diseases such as systemic lupus erythematosus, rheumatoid arthritis, myositis, Sjögren's syndrome, systemic sclerosis, gout, endometriosis, atopic dermatitis and psoriasis.

Aspects of the specification relating to a medicament include those wherein the medicament is intended for human use.

In a further aspect there is provided a process of making Compound (I) comprising reacting

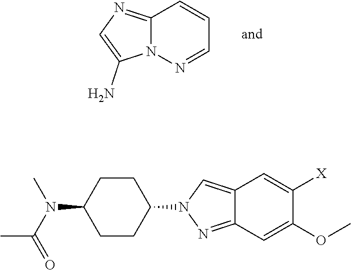

in the presence of carbon monoxide and a catalyst, wherein the group X is selected from Br, Cl, I, OTf and OSO₂Me.

In a further aspect the present specification provides N-((1r,4r)-4-(5-bromo-6-methoxy-2H-indazol-2-yl)cyclohexyl)-N-methylacetamide

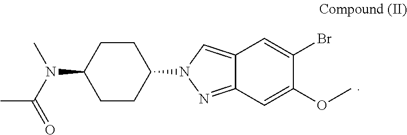 [SMILES: COc1cc2nn([C@H]3CC[C@H](N(C)C(C)=O)CC3)cc2cc1Br]

So that the specification may be fully understood, reference to the following Figures are made herein.

FIG. 1 X-ray powder diffraction pattern of Compound (I) Form A, an anhydrous physical form of N-(Imidazo[1,2-b]pyridazin-3-yl)-6-methoxy-2-((1r,4r)-4-(N-methylacetamido)cyclohexyl)-2H-indazole-5-carboxamide.

FIG. 2 X-ray powder diffraction pattern of Compound (I) Form B, a trihydrate physical form of N-(Imidazo[1,2-b]pyridazin-3-yl)-6-methoxy-2-((1r,4r)-4-(N-methylacetamido)cyclohexyl)-2H-indazole-5-carboxamide.

FIG. 3 X-ray powder diffraction pattern of Compound (I) Form C, a dihydrate physical form of N-(Imidazo[1,2-b]pyridazin-3-yl)-6-methoxy-2-((1r,4r)-4-(N-methylacetamido)cyclohexyl)-2H-indazole-5-carboxamide.

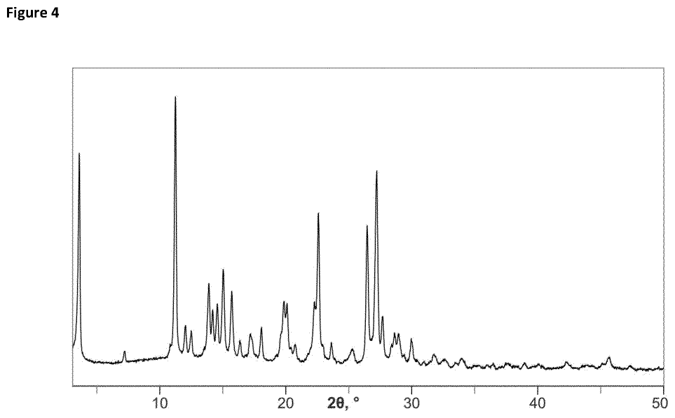

FIG. 4 X-ray powder diffraction pattern of Compound (I) oxalate form, a physical form of N-(Imidazo[1,2-b]pyridazin-3-yl)-6-methoxy-2-((1r,4r)-4-(N-methylacetamido)cyclohexyl)-2H-indazole-5-carboxamide.

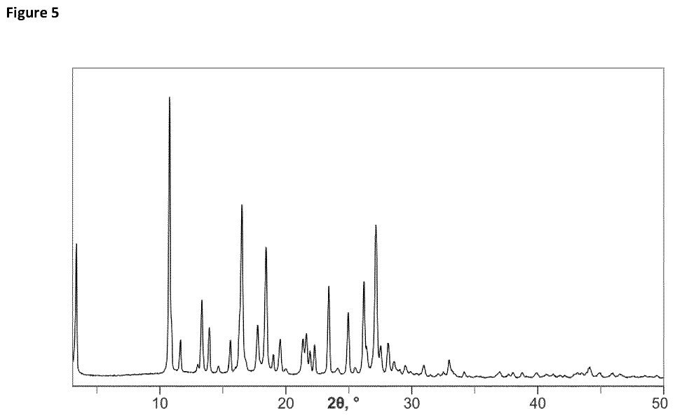

FIG. 5 X-ray powder diffraction pattern of Compound (I) 3-hydroxybenzoic acid form, a physical form of N-(Imidazo[1,2-b]pyridazin-3-yl)-6-methoxy-2-((1r,4r)-4-(N-methylacetamido)cyclohexyl)-2H-indazole-5-carboxamide.

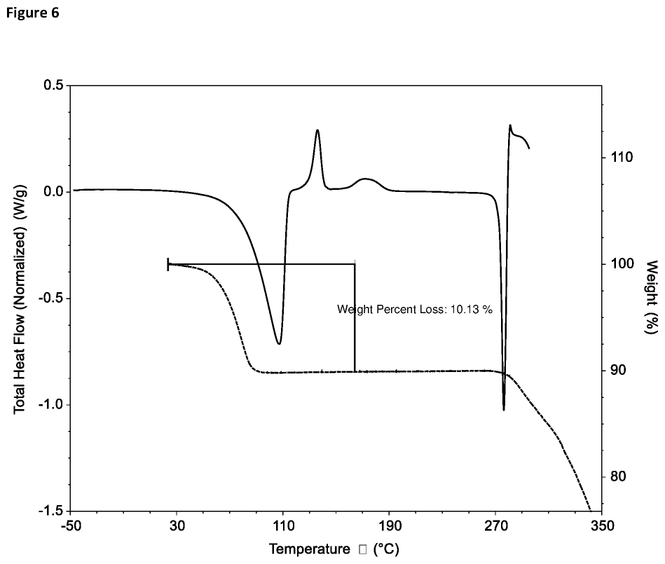

FIG. 6 Thermogravimetric analysis/Differential Scanning calorimetry Plot of trihydrate Form B reveals that controlled heating causes dehydration to an amorphous anhydrous form that proceeds to crystallise to anhydrous Form D on further heating. Continued heating of Form D causes conversion into Form A. Solid and dashed lines represent Differential Scanning calorimetry and Themogravimetric analysis, respectively.

In a first embodiment the specification provides a crystalline form of N-(Imidazo[1,2-b]pyridazin-3-yl)-6-methoxy-2-((1r,4r)-4-(N-methylacetamido)cyclohexyl)-2H-indazole-5-carboxamide (Compound (I)) or a pharmaceutically acceptable salt or solvate thereof:

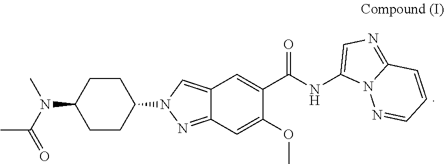 [SMILES: COc1cc2nn([C@H]3CC[C@H](N(C)C(C)=O)CC3)cc2cc1C(=O)Nc1cnc2cccnn12]

In embodiments the crystalline form of Compound (I) is an anhydrous crystalline form. In one such embodiment the crystalline form is Compound (I) Form A and is characterised in providing at least one of the following 20 values measured using Cu K_{α} radiation: 4.9° and 23.4. Further characteristics of Form A are described herein below.

In embodiments the crystalline form of Compound (I) is an hydrate crystalline form. In one such embodiment the crystalline form is Compound (I) trihydrate Form B and is characterised in providing at least one of the following 20 values measured using Cu K_{α} radiation: 17.2° and 26.1°. Further characteristics of Form B are described herein below.

In embodiments there is provided a salt form of Compound (I). In one such embodiment there is provided an oxalate salt form of Compound (I) that has a crystalline form characterised in providing at least one of the following 20 values measured using Cu K_{α} radiation: 11.2° and 27.2°. Further characteristics of Compound (I) oxalate salt form are described herein below.

In embodiments there is provided a co-crystal form of Compound (I). In one such embodiment there is provided Compound (I) 3-hydroxybenzoic acid crystalline form that is characterised in providing at least one of the following 20 values measured using Cu K_{α} radiation: 10.8° and 16.5°. Further characteristics of Compound (I) 3-hydroxybenzoic acid physical form are described herein below.

In embodiments of the present specification there is provided a process for making Compound (I) comprising reacting a compound of Formula (A), imidazo[1,2-b]pyridazin-3-amine dissolved in a suitable solvent with carbon monoxide. The group X in the compound of Formula (A) is a leaving group, for example a leaving group selected from Br, Cl, I, OSO₂R wherein R is a methyl, trifluoromethyl or tolyl. The reaction is conveniently performed with a palladium catalyst, for example a palladium (II) catalyst. The palladium (II) catalyst may be a palladium (II) catalyst featuring a diphosphine ligand such as Pd(dppf)Cl₂.

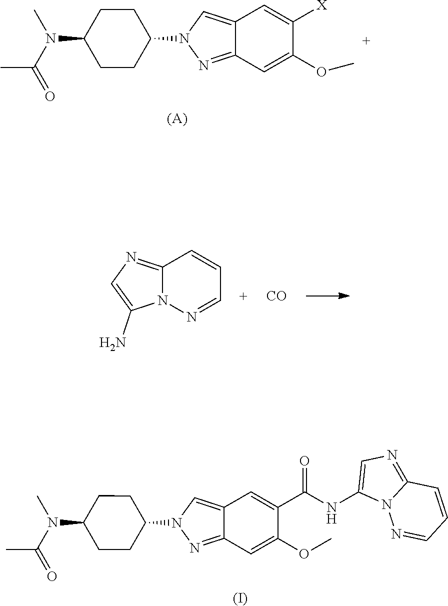

### Form A

Compound (I) form A, an anhydrous physical form of N-(Imidazo[1,2-b]pyridazin-3-yl)-6-methoxy-2-((1r,4r)-4-(N-methylacetamido)cyclohexyl)-2H-indazole-5-carboxamide, is characterised in providing at least one of the following 20 values measured using Cu K_{α} radiation: 4.9° and 23.4°. Compound (I) Form A is characterised in providing an X-ray powder diffraction pattern, substantially as shown in FIG. 1. The ten most prominent peaks are shown in Table 1:

TABLE 1
Ten most prominent peaks of X-ray powder diffraction
pattern of Compound (I) Form A.

| 2θ, ° | Relative intensity |
|---|---|
| 4.9 | very strong |
| 9.7 | strong |
| 12.5 | very strong |
| 16.7 | very strong |
| 18.8 | very strong |
| 19.5 | very strong |
| 20.7 | very strong |
| 23.4 | very strong |
| 25.1 | very strong |
| 27.4 | very strong |

According to the present specification there is provided a crystalline form, Compound (I) Form A, which has an X-ray powder diffraction pattern with at least one specific peak at about 2-theta=4.9°.

According to the present specification there is provided a crystalline form, Compound (I) Form A, which has an X-ray powder diffraction pattern with at least one specific peak at about 2-theta=23.4°.

According to the present specification there is provided a crystalline form, Compound (I) Form A, which has an X-ray powder diffraction pattern with at least two specific peaks at about 2-theta=4.9 and 23.4°.

According to the present specification there is provided a crystalline form, Compound (I) Form A, which has an X-ray powder diffraction pattern with specific peaks at about 2-theta=4.9, 12.5, 16.7, 18.8, and 23.4°.

According to the present specification there is provided a crystalline form, Compound (I) Form A, which has an X-ray powder diffraction pattern with specific peaks at about 2-theta=4.9, 9.7, 12.5, 16.7, 18.8, 19.5, 20.7, 23.4, 25.1, and 27.4°.

According to the present specification there is provided Compound (I) Form A which has an X-ray powder diffraction pattern substantially the same as the X-ray powder diffraction pattern shown in FIG. 1.

According to the present specification there is provided a crystalline form, Compound (I) Form A, wherein said has an X-ray powder diffraction pattern with at least one specific peak at 2-theta=4.9° plus or minus 0.2° 2-theta.

According to the present specification there is provided a crystalline form, Compound (I) Form A, wherein said has an X-ray powder diffraction pattern with at least one specific peak at 2-theta=23.4° plus or minus 0.2° 2-theta.

According to the present specification there is provided a crystalline form, Compound (I) Form A, wherein said has an X-ray powder diffraction pattern with at least two specific peaks at 2-theta=4.9 and 23.4° wherein said values may be plus or minus 0.2° 2-theta.

According to the present specification there is provided a crystalline form, Compound (I) Form A, wherein said has an X-ray powder diffraction pattern with specific peaks at about 2-theta=4.9, 12.5, 16.7 18.8, and 23.4° plus or minus 0.2° 2-theta.

According to the present specification there is provided a crystalline form, Compound (I) Form A, wherein said has an X-ray powder diffraction pattern with specific peaks at about 2-theta=4.9, 9.7, 12.5, 16.7, 18.8, 19.5, 20.7, 23.4, 25.1, and 27.4° plus or minus 0.2° 2-theta.

When it is stated that the present specification relates to a crystalline form of Compound (I) Form A, the degree of crystallinity is conveniently greater than about 60%, more conveniently greater than about 80%, preferably greater than about 90% and more preferably greater than about 95%. Most preferably the degree of crystallinity is greater than about 98%.

The Compound (I) Form A provides X-ray powder diffraction patterns substantially the same as the X-ray powder diffraction patterns shown in FIG. 1 and has substantially the ten most prominent peaks (angle 2-theta values) shown in Table 1. It will be understood that the 2-theta values of the X-ray powder diffraction pattern may vary slightly from one machine to another or from one sample to another, and so the values quoted are not to be construed as absolute.

It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions. Therefore it should be understood that the Compound (I) Form A of the present specification is not limited to the crystals that provide X-ray powder diffraction patterns identical to the X-ray powder diffraction pattern shown in FIG. 1, and any crystals providing X-ray powder diffraction patterns substantially the same as those shown in FIG. 1 fall within the scope of the present specification. A person skilled in the art of X-ray powder diffraction is able to judge the substantial identity of X-ray powder diffraction patterns.

### Form B

Compound (I) Form B, a trihydrate physical form of N-(Imidazo[1,2-b]pyridazin-3-yl)-6-methoxy-2-((1r,4r)-4-(N-methylacetamido)cyclohexyl)-2H-indazole-5-carboxamide, is characterised in providing at least one of the following 20 values measured using Cu K_{α} radiation: 17.2° and 26.1°. Compound (I) Form B is characterised in providing an X-ray powder diffraction pattern, substantially as shown in FIG. 2. The ten most prominent peaks are shown in Table 2:

TABLE 1
Ten most prominent peaks of X-ray powder diffraction
pattern of Compound (I) Form B.

| 2θ, ° | Relative intensity |
|---|---|
| 6.7 | very strong |
| 11.3 | very strong |
| 11.9 | very strong |
| 12.3 | strong |
| 15.7 | strong |
| 17.2 | very strong |
| 22.7 | strong |
| 26.1 | very strong |
| 27.2 | strong |
| 28.5 | strong |

According to the present specification there is provided a crystalline form, Compound (I) Form B, which has an X-ray powder diffraction pattern with at least one specific peak at about 2-theta=17.2°.

According to the present specification there is provided a crystalline form, Compound (I) Form B, which has an X-ray powder diffraction pattern with at least one specific peak at about 2-theta=26.1°.

According to the present specification there is provided a crystalline form, Compound (I) Form B, which has an X-ray powder diffraction pattern with at least two specific peaks at about 2-theta=17.2° and 26.1°.

According to the present specification there is provided a crystalline form, Compound (I) Form B, which has an X-ray powder diffraction pattern with specific peaks at about 2-theta=6.7, 11.3, 11.9, 17.2, and 26.1°.

According to the present specification there is provided a crystalline form, Compound (I) Form B, which has an X-ray powder diffraction pattern with specific peaks at about 2-theta=6.7, 11.3, 11.9, 12.3, 15.7, 17.2, 22.7, 26.1, 27.2, and 28.5°.

According to the present specification there is provided Compound (I) Form B which has an X-ray powder diffraction pattern substantially the same as the X-ray powder diffraction pattern shown in FIG. 2.

According to the present specification there is provided a crystalline form, Compound (I) Form B, wherein said has an X-ray powder diffraction pattern with at least one specific peak at 2-theta=17.2° plus or minus 0.2° 2-theta.

According to the present specification there is provided a crystalline form, Compound (I) Form B, wherein said has an X-ray powder diffraction pattern with at least one specific peak at 2-theta=26.1° plus or minus 0.2° 2-theta.

According to the present specification there is provided a crystalline form, Compound (I) Form B, wherein said has an X-ray powder diffraction pattern with at least two specific peaks at 2-theta=17.2° and 26.1° wherein said values may be plus or minus 0.2° 2-theta.

According to the present specification there is provided a crystalline form, Compound (I) Form B, wherein said has an X-ray powder diffraction pattern with specific peaks at about 2-theta=6.7, 11.3, 11.9, 17.2, 26.1° plus or minus 0.2° 2-theta.

According to the present specification there is provided a crystalline form, Compound (I) Form B, wherein said has an X-ray powder diffraction pattern with specific peaks at about 2-theta=6.7, 11.3, 11.9, 12.3, 15.7, 17.2, 22.7, 26.1, 27.2, and 28.5° plus or minus 0.2° 2-theta.

When it is stated that the present specification relates to a crystalline form of Compound (I) Form B, the degree of crystallinity is conveniently greater than about 60%, more conveniently greater than about 80%, preferably greater than about 90% and more preferably greater than about 95%. Most preferably the degree of crystallinity is greater than about 98%.

The Compound (I) Form B provides X-ray powder diffraction patterns substantially the same as the X-ray powder diffraction patterns shown in FIG. 2 and has substantially the ten most prominent peaks (angle 2-theta values) shown in Table 1. It will be understood that the 2-theta values of the X-ray powder diffraction pattern may vary slightly from one machine to another or from one sample to another, and so the values quoted are not to be construed as absolute.

It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions. Therefore it should be understood that the Compound (I) Form B of the present specification is not limited to the crystals that provide X-ray powder diffraction patterns identical to the X-ray powder diffraction pattern shown in FIG. 2, and any crystals providing X-ray powder diffraction patterns substantially the same as those shown in FIG. 2 fall within the scope of the present specification. A person skilled in the art of X-ray powder diffraction is able to judge the substantial identity of X-ray powder diffraction patterns.

### Form C

A further crystalline form of Compound (I), namely a meta stable dihydrate form of N-(Imidazo[1,2-b]pyridazin-3-yl)-6-methoxy-2-((1r,4r)-4-(N-methylacetamido)cyclohexyl)-2H-indazole-5-carboxamide, Form C, was also obtained. Compound (I) Form C provides X-ray powder diffraction patterns substantially the same as the X-ray powder diffraction patterns shown in FIG. 3.

### Compound (I) Oxalate Salt Form

Compound (I) oxalate crystalline form is a crystalline salt form comprising a 1:1 ratio of N-(Imidazo[1,2-b]pyridazin-3-yl)-6-methoxy-2-((1r,4r)-4-(N-methylacetamido)cyclohexyl)-2H-indazole-5-carboxamide and oxalic acid that is characterised in providing at least one of the following 20 values measured using Cu K_{α} radiation: 11.2° and 27.2°. Compound (I) oxalate crystalline form is characterised in providing an X-ray powder diffraction pattern, substantially as shown in FIG. 4. The ten most prominent peaks are shown in Table 3:

TABLE 3
Ten most prominent peaks of X-ray powder diffraction
pattern of Compound (I) oxalate crystalline form.

| 2θ, ° | Relative intensity |
|---|---|
| 3.6 | very strong |
| 11.2 | very strong |
| 13.9 | very strong |
| 14.6 | strong |
| 15.0 | very strong |
| 15.7 | very strong |
| 18.1 | strong |
| 22.6 | very strong |
| 26.5 | very strong |
| 27.2 | very strong |

According to the present specification there is provided a crystalline form, Compound (I) oxalate form, which has an X-ray powder diffraction pattern with at least one specific peak at about 2-theta=11.2°.

According to the present specification there is provided a crystalline form, Compound (I) oxalate form, which has an X-ray powder diffraction pattern with at least one specific peak at about 2-theta=27.2°.

According to the present specification there is provided a crystalline form, Compound (I) oxalate form, which has an X-ray powder diffraction pattern with at least two specific peaks at about 2-theta=11.2 and 27.2°.

According to the present specification there is provided a crystalline form, Compound (I) oxalate form, which has an X-ray powder diffraction pattern with specific peaks at about 2-theta=11.2, 27.2, 3.6, 22.6 and 26.5°.

According to the present specification there is provided a crystalline form, Compound (I) oxalate form, which has an X-ray powder diffraction pattern with specific peaks at about 2-theta=11.2, 27.2, 3.6, 22.6, 26.5, 15.0, 13.9, 15.7, 14.6 and 18.1°.

According to the present specification there is provided Compound (I) oxalate form, which has an X-ray powder diffraction pattern substantially the same as the X-ray powder diffraction pattern shown in FIG. 4.

According to the present specification there is provided a crystalline form, Compound (I) oxalate form, wherein said has an X-ray powder diffraction pattern with at least one specific peak at 2-theta=11.2° plus or minus 0.2° 2-theta.

According to the present specification there is provided a crystalline form, Compound (I) oxalate form, wherein said has an X-ray powder diffraction pattern with at least one specific peak at 2-theta=27.2° plus or minus 0.2° 2-theta.

According to the present specification there is provided a crystalline form, Compound (I) oxalate form, wherein said has an X-ray powder diffraction pattern with at least two specific peaks at 2-theta=11.2 and 27.2° wherein said values may be plus or minus 0.2° 2-theta.

According to the present specification there is provided a crystalline form, Compound (I) oxalate form, wherein said has an X-ray powder diffraction pattern with specific peaks at about 2-theta=11.2, 27.2, 3.6, 22.6 and 26.5° plus or minus 0.2° 2-theta.

According to the present specification there is provided a crystalline form, Compound (I) oxalate form, wherein said has an X-ray powder diffraction pattern with specific peaks at about 2-theta=11.2, 27.2, 3.6, 22.6, 26.5, 15.0, 13.9, 15.7, 14.6 and 18.1° plus or minus 0.2° 2-theta.

When it is stated that the present specification relates to a crystalline form of Compound (I) oxalate form, the degree of crystallinity is conveniently greater than about 60%, more conveniently greater than about 80%, preferably greater than about 90% and more preferably greater than about 95%. Most preferably the degree of crystallinity is greater than about 98%.

The Compound (I) oxalate form X-ray powder diffraction patterns substantially the same as the X-ray powder diffraction patterns shown in FIG. 4 and has substantially the ten most prominent peaks (angle 2-theta values) shown in Table. It will be understood that the 2-theta values of the X-ray powder diffraction pattern may vary slightly from one machine to another or from one sample to another, and so the values quoted are not to be construed as absolute.

It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions. Therefore it should be understood that the Compound (I) oxalate form of the present specification is not limited to the crystals that provide X-ray powder diffraction patterns identical to the X-ray powder diffraction pattern shown in FIG. 4, and any crystals providing X-ray powder diffraction patterns substantially the same as those shown in FIG. 4 fall within the scope of the present specification. A person skilled in the art of X-ray powder diffraction is able to judge the substantial identity of X-ray powder diffraction patterns.

### Compound (I) 3-Hydroxybenzoic Acid Form

Compound (I) 3-hydroxybenzoic acid crystalline form is a co-crystal form that contains N-(Imidazo[1,2-b]pyridazin-3-yl)-6-methoxy-2-((1r,4r)-4-(N-methylacetamido)cyclohexyl)-2H-indazole-5-carboxamide and 3-hydroxybenzoic acid in a 1:1 ratio that is characterised in providing at least one of the following 20 values measured using Cu K_{α} radiation: 10.8° and 16.5°. Compound (I) 3-hydroxybenzoic acid form is characterised in providing an X-ray powder diffraction pattern, substantially as shown in FIG. 5. The ten most prominent peaks are shown in Table 1. It is possible that this co-crystal form is a salt form, but this has not been formally determined.

TABLE 4
Ten most prominent peaks of X-ray powder diffraction
pattern of Compound (I) 3-hydroxybenzoic acid.

| 2θ, ° | Relative intensity |
|---|---|
| 3.4 | very strong |
| 10.8 | very strong |
| 13.3 | very strong |
| 13.9 | strong |
| 16.5 | very strong |
| 17.8 | very strong |
| 18.4 | very strong |
| 23.4 | very strong |
| 24.9 | very strong |
| 27.1 | very strong |

According to the present specification there is provided a crystalline form, Compound (I) 3-hydroxybenzoic acid, which has an X-ray powder diffraction pattern with at least one specific peak at about 2-theta=10.8°.

According to the present specification there is provided a crystalline form, Compound (I) 3-hydroxybenzoic acid, which has an X-ray powder diffraction pattern with at least one specific peak at about 2-theta=16.5°.

According to the present specification there is provided a crystalline form, Compound (I) 3-hydroxybenzoic acid, which has an X-ray powder diffraction pattern with at least two specific peaks at about 2-theta=10.8° and 16.5°.

According to the present specification there is provided a crystalline form, Compound (I) 3-hydroxybenzoic acid, which has an X-ray powder diffraction pattern with specific peaks at about 2-theta=10.8, 16.5, 27.1, 18.4 and 3.4°.

According to the present specification there is provided a crystalline form, Compound (I) 3-hydroxybenzoic acid, which has an X-ray powder diffraction pattern with specific peaks at about 2-theta=10.8, 16.5, 27.1, 18.4, 3.4, 23.4, 13.3, 24.9, 17.8 and 13.9°.

According to the present specification there is provided Compound (I) 3-hydroxybenzoic acid which has an X-ray powder diffraction pattern substantially the same as the X-ray powder diffraction pattern shown in FIG. 5.

According to the present specification there is provided a crystalline form, Compound (I) 3-hydroxybenzoic acid, wherein said has an X-ray powder diffraction pattern with at least one specific peak at 2-theta=10.8° plus or minus 0.2° 2-theta.

According to the present specification there is provided a crystalline form, Compound (I) 3-hydroxybenzoic acid, wherein said has an X-ray powder diffraction pattern with at least one specific peak at 2-theta=16.5° plus or minus 0.2° 2-theta.

According to the present specification there is provided a crystalline form, Compound (I) 3-hydroxybenzoic acid, wherein said has an X-ray powder diffraction pattern with at least two specific peaks at 2-theta=10.8° and 16.5° wherein said values may be plus or minus 0.2° 2-theta.

According to the present specification there is provided a crystalline form, Compound (I) 3-hydroxybenzoic acid, wherein said has an X-ray powder diffraction pattern with specific peaks at about 2-theta=10.8, 16.5, 27.1, 18.4 and 3.4° plus or minus 0.2° 2-theta.

According to the present specification there is provided a crystalline form, Compound (I) 3-hydroxybenzoic acid, wherein said has an X-ray powder diffraction pattern with specific peaks at about 2-theta=10.8, 16.5, 27.1, 18.4, 3.4, 23.4, 13.3, 24.9, 17.8 and 13.9° plus or minus 0.2° 2-theta.

When it is stated that the present specification relates to a crystalline form of Compound (I) 3-hydroxybenzoic acid, the degree of crystallinity is conveniently greater than about 60%, more conveniently greater than about 80%, preferably greater than about 90% and more preferably greater than about 95%. Most preferably the degree of crystallinity is greater than about 98%.

The Compound (I) 3-hydroxybenzoic acid provides X-ray powder diffraction patterns substantially the same as the X-ray powder diffraction patterns shown in FIG. 5 and has substantially the ten most prominent peaks (angle 2-theta values) shown in Table 1. It will be understood that the 2-theta values of the X-ray powder diffraction pattern may vary slightly from one machine to another or from one sample to another, and so the values quoted are not to be construed as absolute.

It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions. Therefore it should be understood that the Compound (I) 3-hydroxybenzoic acid of the present specification is not limited to the crystals that provide X-ray powder diffraction patterns identical to the X-ray powder diffraction pattern shown in FIG. 5, and any crystals providing X-ray powder diffraction patterns substantially the same as those shown in FIG. 5 fall within the scope of the present specification. A person skilled in the art of X-ray powder diffraction is able to judge the substantial identity of X-ray powder diffraction patterns.

### Anhydrous Form D

A sample of the trihydrate Form B was subjected to thermal gravimetric analysis (TGA) and differential scanning calorimetry (DSC), the results of which are presented in FIG. 6. Briefly, TGA and DSC measurements were performed using a TG Discovery 550 (TA instruments, Germany) and DSC Discovery 2500 (TA instruments, Germany), respectively. Approximately 5 mg for TGA and 2-3 mg of the sample for DSC were weighed into an aluminium pan. The samples were then heated from room temperature to 350° C. for TGA and from −50° C. to 300° C. for DSC, with a heating rate of 3° C./min under a nitrogen purge of 100 mL/min. An empty aluminium pan was used as a reference for DSC. Open and closed pans were used for TGA and DSC measurements, respectively. Modulated mode was used for DSC measurement with modulation temperature amplitude and modulation period set at 1° C. and 60 s, respectively.

In the TGA (dashed line of figure, TGA scale on right hand side) a weight loss due of 10.13% was observed over the temperature range of from below 25° C. to around 120° C. This weight loss is due to dehydration, with the 10.13% weight loss being equivalent with 2.89 water molecules per molecule of API. This matches with the formal assignment of Form B as a trihydrate. The dehydration proceeds from relatively low temperature, i.e. below 25° C. The DSC measurement (solid line, DSC scale on left hand side) was performed starting from −50° C. As can be seen from FIG. 6 the dehydration concludes by around 120° C. After dehydration, an exotherm was observed with an onset around 130° C. indicating crystallization and resulted the new anhydrous Form D. Another exothermic curve was observed with the onset at 160° C. indicating crystallization to Form A.

### In-Situ Characterization of Form D

In order to learn more about the characteristics of Form D, characterization was performed in-situ using a SmartLab X-ray diffractometer with equipped with DSC attachment (Rigaku, Japan). A sample of Form B was placed on a flat aluminum DSC pan. The PXRD pattern was collected from 2θ=5° to 38° with a step and scan speed of 0.01° and 10° min⁻¹, respectively. The heating speed for DSC was set at 1° C. min⁻¹. A Cu K_{α} source was employed with the X-ray power was set to 40 kV, 50 mA. The heating process was continued until full dehydration of Form B to amorphous form followed by crystallization as indicated by the arising diffraction peaks. The heating process was stopped immediately after second PXRD pattern of crystalline material was obtained (at ca 111° C.). The furnace was subsequently cooled to room temperature with a ramping speed of 10° C. min⁻¹. The material was then analyzed with electron diffraction for crystal structure determination. Electron diffraction measurements were collected using a Rigaku Synergy-ED (Rigaku, Japan) equipped with a Rigaku HyPix-ED detector optimized for operation in the Micro-ED experimental setup. A total of two data sets were collected and merged resulting in a comprehensive data set with a resolution limit of 1.05 Å. Form D was determined to have crystallized in the triclinic space group P-1 with the following lattice parameters: a=6.995 (7) Å, b=11.416 (12) Å, c=15.90 (2) Å, a=71.25 (10) ‘, 6=85.46 (10)°, γ=89.23 (9), V=1198 (2) Å³ with Z and Z’ were found to be 2 and 1, respectively.

### X-ray Powder Diffraction Analysis

The X-ray powder diffraction analysis was performed according to standard methods, which can be found in e.g. Kitaigorodsky, A. I. (1973), Molecular Crystals and Molecules, Academic Press, New York; Bunn, C. W. (1948), Chemical Crystallography, Clarendon Press, London; or Klug, H. P. & Alexander, L. E. (1974), X-ray Diffraction Procedures, John Wiley & Sons, New York.

Persons skilled in the art of X-ray powder diffraction will realize that the relative intensity of peaks can be affected by, for example, grains above 30 microns in size and non-unitary aspect ratios, which may affect analysis of samples. The skilled person will also realize that the position of reflections can be affected by the precise height at which the sample sits in the diffractometer and the zero calibration of the diffractometer. The surface planarity of the sample may also have a small effect. Hence the diffraction pattern data presented are not to be taken as absolute values. (Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures).

Generally, a measurement error of a diffraction angle in an X-ray powder diffractogram is about 5% or less, in particular plus or minus 0.2° 2-theta, and such degree of a measurement error should be taken into account when considering the X-ray powder diffraction patterns in FIG. 1, FIG. 2, FIG. 3, FIG. 4 and FIG. 5 and when reading Table 1, Table 2, Table 3 and Table 4. Furthermore, it should be understood that intensities may fluctuate depending on experimental conditions and sample preparation (preferred orientation). Definition of relative intensity is described in Table 3.

X-ray powder diffraction data was measured with Corundum as an internal reference. The X-ray powder diffraction (referred to herein as XRPD) pattern was determined by mounting a sample on a zero background holder single silicon crystal and spreading out the sample into a thin layer.

The powder X-ray diffraction was recorded with a theta-two theta scan axis and in one dimensional scan with Rigaku Miniflex 600 (wavelength of X-rays 1.5418 Å nickel-filtered Cu K_{α} radiation, 40 kV, 15 mA) equipped with D/Tex Ultra detector. Automatic variable anti scattering slits were used and the samples were rotated at 30 revolution per minute during measurement. Samples were scanned from 3-50° 2-theta using a 0.01° and 1°/min step width and scan speed respectively.

The X-ray powder diffraction analysis was performed according to standard methods, which can be found in e.g. Kitaigorodsky, A. I. (1973), Molecular Crystals and Molecules, Academic Press, New York; Bunn, C. W. (1948), Chemical Crystallography, Clarendon Press, London; or Klug, H. P. & Alexander, L. E. (1974), X-ray diffraction procedures for polycrystalline and amorphous materials, John Wiley, New York, London.

TABLE 2
Definition of relative intensity

| % Relative Intensity* | Definition |
|---|---|
| 25-100 | vs (very strong) |
| 10-25 | s (strong) |
| 3-10 | m (medium) |
| 1-3 | w (weak) |

*The relative intensities are derived from diffractograms measured with fixed slits

A person skilled in the art understands that the value or range of values observed in a particular compound's DSC Thermogram will show variation between batches of different purities. Therefore, whilst for one compound the range may be small, for others the range may be quite large. Generally, a measurement error of a diffraction angle in DSC thermal events is approximately plus or minus 5° C., and such degree of a measurement error should be taken into account when considering the DSC data included herein.

Crystallisation of the desired form in a process described herein may be aided by seeding with crystals of the desired form. The seed crystals may be obtained using one of the methods described in the Examples. The use of seeding is particularly advantageous in larger-scale manufacture.

In the present specification in instances where the compound is described as having a “X-ray powder diffraction pattern with at least one specific peak at 2θ about = . . . ” the XRPD of the compound may contain one or more of the 2θ values listed. For example one or more of the 2θ values, 2 or more of the 2θ values or 3 or more of the 2θ values listed.

In the preceding paragraphs defining the X-ray powder diffraction peaks for the crystalline form of Compound (I), the term “about=” is used in the expression” . . . at 20 about= . . . “to indicate that the precise position of peaks (i.e. the recited 2-theta angle values) should not be construed as being absolute values because, as will be appreciated by those skilled in the art, the precise position of the peaks may vary slightly between one measurement apparatus and another, from one sample to another, or as a result of slight variations in measurement conditions utilised. It is also stated in the preceding paragraphs that the crystalline form of Compound (I) provide X-ray powder diffraction patterns ‘substantially’ the same as the X-ray powder diffraction patterns shown in FIG. 1 has substantially the most prominent peaks (2-theta angle values) shown in Table 1. It is to be understood that the use of the term ‘substantially’ in this context is also intended to indicate that the 2-theta angle values of the X-ray powder diffraction patterns may vary slightly from one apparatus to another, from one sample to another, or as a result of slight variations in measurement conditions utilised, so the peak positions shown in the Figure or quoted in the Table are again not to be construed as absolute values.

The person skilled in the art of X-ray powder diffraction will realize that the relative intensity of peaks can be affected by, for example, grains above approximately 30 micrometer in size and non-unitary aspect ratios which may affect analysis of samples. Furthermore, it should be understood that intensities may fluctuate depending on experimental conditions and sample preparation such as preferred orientation of the particles in the sample. The use of automatic or fixed divergence slits will also influence the relative intensity calculations. A person skilled in the art can handle such effects when comparing diffraction patterns.

The person skilled in the art of X-ray powder diffraction will also realize that due to difference in sample heights and errors in the calibration of the detector position, a small shift in the 20 positions could occur. Generally, a difference of ±0.1° from the given value are to be considered correct.

Compound (I) forms described herein may also be characterised and/or distinguished from other physical forms using other suitable analytical techniques, for example NIR spectroscopy or solid-state nuclear magnetic resonance spectroscopy.

The chemical structure of Compound (I) forms described herein can be confirmed by routine methods for example proton nuclear magnetic resonance (NMR) analysis.

Compound (I) forms may be prepared as described in the Examples hereinafter.

### Process

One synthesis of Compound (I) is described in PCT/EP2021/084916.

The present specification provides a process for making Compound (I) comprising reacting a compound of Formula (A), imidazo[1,2-b]pyridazin-3-amine dissolved in a suitable solvent under an atmosphere of carbon monoxide with a suitable catalyst. The reaction may be performed under a high pressure of carbon monoxide, for example a pressure of 5 atmospheres or more, for example 15 atmospheres. The group X in the compound of Formula (A) is a leaving group, for example a leaving group selected from Br, Cl, I, OSO₂R wherein R is a methyl, trifluoromethyl or tolyl. In embodiments the group X is Br. The reaction is conveniently performed with a palladium catalyst, for example a palladium (II) catalyst. The palladium (II) catalyst may be a palladium (II) catalyst featuring a diphosphine ligand such as dppf (1,1′-bis(diphenylphosphino) ferrocene), for example Pd(dppf)Cl₂. Other suitable palladium (II) catalyst systems maybe used such as those featuring a ligand such as Xantphos ((9,9-Dimethyl-9H-xanthene-4,5-diyl)bis(diphenylphosphane)), BINAP ((2,2′-bis(diphenylphosphino)-1,1′-binaphthyl)) or dppp (1,3-bis(diphenylphosphino) propane). The catalyst may be pre-formed or may be generated in situ by stirring a palladium (II) source such as palladium (II) acetate or palladium (II) chloride and an appropriate ligand in an appropriate solvent. Details on palladium catalysed carbonylation are provided in e.g. Brennfuhrer et al, Angew. Chem. Int. Ed 2009, 48, 4114-33. The solvent may be acetonitrile. Other solvents or mixtures of solvents may equally be used.

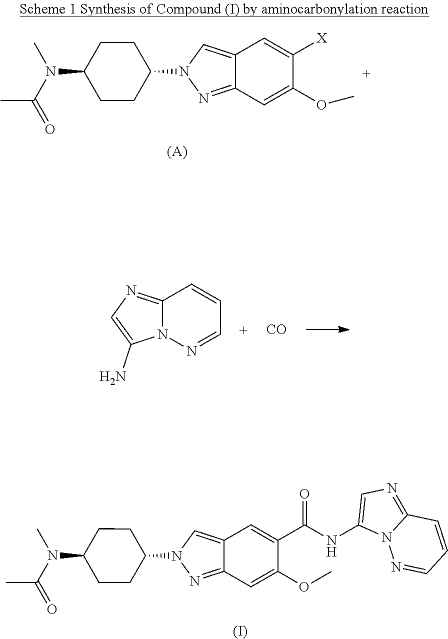

In one practical example, the reaction is performed with N-((1r,4r)-4-(5-bromo-6-methoxy-2H-indazol-2-yl)cyclohexyl)-N-methylacetamide

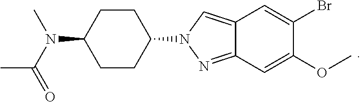 [SMILES: COc1cc2nn([C@H]3CC[C@H](N(C)C(C)=O)CC3)cc2cc1Br]

### Medical and Pharmaceutical Use

A crystalline form of compound (I) may be useful in the prevention or treatment of respiratory diseases such as asthma and chronic obstructive pulmonary disease (COPD), of cancer, of inflammatory diseases and of autoinflammatory/autoimmune diseases such as systemic lupus erythematosus, rheumatoid arthritis, myositis, Sjögren's syndrome, systemic sclerosis, gout, endometriosis, atopic dermatitis and psoriasis in a mammal, particularly a human.

### Combination Therapy

A crystalline form of compound (I) may also be administered in conjunction with other compounds used for the treatment of the above conditions.

In another embodiment, there is a combination therapy wherein a compound of a crystalline form of Compound (I), and a second active ingredient are administered concurrently, sequentially or in admixture, for the treatment of one or more of the conditions listed above. Such a combination may be used in combination with one or more further active ingredients.

### Administration

There is provided a method of treatment of a condition where inhibition of IRAK4 is required, which method comprises administration of a therapeutically effective amount of a crystalline form of compound (I) to a person suffering from, or susceptible to, such a condition.

A crystalline form of compound (I), will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient in a pharmaceutically acceptable dosage form. Depending upon the disorder and patient to be treated and the route of administration, the compositions may be administered at varying doses.

Dosage forms suitable for oral use form one aspect of the specification.

The compositions of the specification may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, granulating and disintegrating agents such as corn starch; binding agents such as starch; lubricating agents such as magnesium stearate. Tablet formulations may be uncoated or coated using conventional coating agents and procedures well known in the art.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration.

Suitable daily doses of a crystalline form of compound (I), in therapeutic treatment of humans are about 0.0001-100 mg/kg body weight.

Oral formulations are preferred particularly tablets or capsules which may be formulated by methods known to those skilled in the art to provide doses of the active compound in the range of 0.1 mg to 1000 mg.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

According to a further aspect, there is thus provided a pharmaceutical composition including a crystalline form of compound (I) in admixture with pharmaceutically acceptable adjuvants, diluents and/or carriers.

### EXAMPLES

Abbreviations used for analytical data, are consistent with the common usage in the field (see J Med Chem Standard Abbreviations and Acronyms http://pubsapp.acs.org/paragonplus/submission/jmcmar/jmcmar_abbreviations.pdf?). The compound names provided below are generated using PerkinElmer ChemDraw Professional, Version 20.0.2.51. In instances where there is uncertainty as to the absolute stereochemistry, relative stereochemistry is specified as far as possible.

### Preparation of N-(Imidazo[1,2-b]pyridazin-3-yl)-6-methoxy-2-((1r,4r)-4-(N-methylacetamido)cyclohexyl)-2H-indazole-5-carboxamide Form A

### 5-Bromo-4-fluoro-2-nitrobenzaldehyde

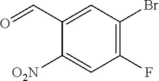 [SMILES: O=Cc1cc(Br)c(F)cc1[N+](=O)[O-]]

A solution of 3-bromo-4-fluorobenzaldehyde (18 kg, 88.6 mol) in concentrated sulfuric acid (54 L) at 0° C. and concentrated nitric acid (17.18 kg, 177.3 mol@65 w/w %) at room temperature (rt) was flowed into a reaction tube at 45° C. for 15 minutes. The mixture was quenched into ice water (180 L) and extracted with toluene (90 L), the organic phase was washed with a 5% aqueous solution of K₃PO₄ (54 L) and then saturated sodium chloride solution (54 L) to give 5-bromo-4-fluoro-2-nitrobenzaldehyde as a solution in toluene which was used without purification in the next step.

### 5-Bromo-4-methoxy-2-nitrobenzaldehyde

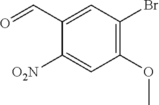

A solution of 5-bromo-4-fluoro-2-nitrobenzaldehyde in toluene (105.3 kg) and sodium methoxide (17.31 kg, 96 mol@30 w/w %) in methanol (110 L) at rt was flowed into a reaction tube at 50° C. for 15 minutes to afford 5-bromo-4-methoxy-2-nitrobenzaldehyde as a solution in toluene/methanol which was used without purification in the next step.

### N-((1r,4r)-4-(5-bromo-6-methoxy-2H-indazol-2-yl)cyclohexyl) acetamide

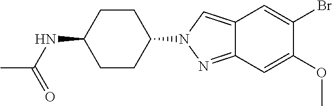 [SMILES: COc1cc2nn([C@H]3CC[C@H](NC(C)=O)CC3)cc2cc1Br]

To a solution of 5-bromo-4-methoxy-2-nitrobenzaldehyde in toluene/methanol (61.47 kg) was added N-[(1R,4R)-4-aminocyclohexyl]acetamide.trifluoroacetic acid salt (3.27 kg, 12.1 mol) and 1,8-Diazabicyclo[5.4.0]undec-7-ene (DBU) (2.81 kg, 18.4 mol), the mixture was stirred at 45° C. for 16 hours and then cooled to rt. Tributylphosphine (8.17 kg, 40.38 mol) was added and the mixture was stirred at 65° C. for 16 hours and then allowed to cool to rt, concentrated and added water (30 L) then extracted with dichloromethane (2×30 L), the organic phase was separated and then concentrated. A solvent swap to isopropylalcohol (IPA) was conducted and the addition of n-heptane afforded a slurry when cooled to 0° C., solids filtered and cake washed with IPA/n-heptane (1:2), dried under vacuum at 50° C. to afford N-((1r,4r)-4-(5-bromo-6-methoxy-2H-indazol-2-yl)cyclohexyl) acetamide (2.19 kg, 5.98 mol).

### N-((1r,4r)-4-(5-bromo-6-methoxy-2H-indazol-2-yl)cyclohexyl)-N-methylacetamide

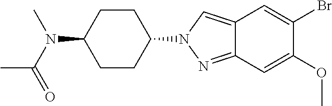 [SMILES: COc1cc2nn([C@H]3CC[C@H](N(C)C(C)=O)CC3)cc2cc1Br]

To a mixture of N-((1r,4r)-4-(5-bromo-6-methoxy-2H-indazol-2-yl)cyclohexyl) acetamide (4.60 kg, 12.56 mol) in THF (46 L) was added Mel (3.39 kg, 23.86 mol) at 0° C. then added tert-potassium butoxide (23.94 mol in THF, volume 13.3 L) dropwise and stirred for 3 hours at 0° C. The mixture was quenched while maintaining the temperature between 0 and 10° C. with acetic acid (907.9 g, 15.12 mol). Cyclopentyl methyl ether was then added (23 L), and the mixture concentrated before another addition of cyclopentyl methyl ether (23 L). The resultant mixture was heated to 100° C. and stirred for 2 hours then cooled to 20° C. over 4 hours before filtering the solids and washing the filter cake with cyclopentyl methyl ether (9.2 L). The isolated solid was dried in a vacuum oven at 40° C. to afford N-((1r,4r)-4-(5-bromo-6-methoxy-2H-indazol-2-yl)cyclohexyl)-N-methylacetamide (4.41 kg, 11.60 mol).

### N-(Imidazo[1,2-b]pyridazin-3-yl)-6-methoxy-2-((1r,4r)-4-(N-methylacetamido)cyclohexyl)-2H-indazole-5-carboxamide

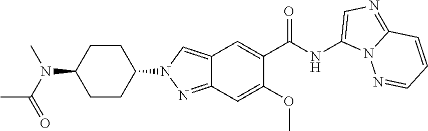 [SMILES: COc1cc2nn([C@H]3CC[C@H](N(C)C(C)=O)CC3)cc2cc1C(=O)Nc1cnc2cccnn12]

N-((1r,4r)-4-(5-bromo-6-methoxy-2H-indazol-2-yl)cyclohexyl)-N-methylacetamide (3.5 kg, 9.20 mol), imidazo[1,2-b]pyridazin-3-amine (1.47 kg, 10.96 mol) and triethylamine (1.86 kg, 18.38 mol) were charged to an autoclave and mixed with acetonitrile (27.3 kg) under nitrogen, stirred for 10 minutes and then treated with Pd(dppf)Cl₂ (0.20 kg, 0.27 mol). The autoclave was sealed and heated at 95° C. for 18 hours under a CO atmosphere at 0.1-0.8 MPa, allowed to cool and filtered, and the filter cake washed with acetonitrile (8.19 kg). (This reaction was repeated at the same scale and the solids combined after filtration). The combined solids were stirred in a mixture of methanol (5.37 kg) and dichloromethane (81.4 kg) and then treated with activated carbon (0.68 kg) and 3-mercaptopropyl ethyl sulfide silica (0.68 kg), stirred at rt for 18 hours then filtered through Celite (3.4 kg) washing the cake with a 10% mixture of methanol and dichloromethane (34.82 kg). The filtrate was filtered through a 0.2 μm filter and then concentrated, diluted with methanol (37.6 kg) when all dichloromethane had been removed, then heated to 50° C. for 2 hours then slowly cooled to 25° C. before centrifugal filtration, the filter cake was washed with methanol (16.12 kg) and dried in a vacuum oven at 50° C. to afford N-(Imidazo[1,2-b]pyridazin-3-yl)-6-methoxy-2-((1r,4r)-4-(N-methylacetamido)cyclohexyl)-2H-indazole-5-carboxamide (6.27 kg, 13.60 mol). A sample of material was retained for seeding purposes in the crystallisation described immediately below.

To a suspension of N-(Imidazo[1,2-b]pyridazin-3-yl)-6-methoxy-2-((1r,4r)-4-(N-methylacetamido)cyclohexyl)-2H-indazole-5-carboxamide (6.03 kg, 13.0 mol) was added methanol (47.8 L) and the mixture agitated and heated to 50° C. To this was added a mixture of methanesulphonic acid (1.25 kg, 13.0 mol) in methanol (3 L) and the solution stirred at 50° C. for 2 hours. N-methyl morpholine (0.131 kg, 1.30 mol) as a solution in methanol (0.6 L) was then added gradually followed by seeding with a sample of N-(Imidazo[1,2-b]pyridazin-3-yl)-6-methoxy-2-((1r,4r)-4-(N-methylacetamido)cyclohexyl)-2H-indazole-5-carboxamide (54.3 g) taken from the 6.27 kg batch of solid prepared directly above. The resultant mixture was stirred overnight at 50° C., treated with a further portion of N-methyl morpholine (1.18 kg, 11.7 mol) in methanol (4.26 kg) and then stirred for 50° C. overnight. The resultant solids were filtered and the cake washed with methanol (2×4.74 kg) and dried to afford N-(Imidazo[1,2-b]pyridazin-3-yl)-6-methoxy-2-((1r,4r)-4-(N-methylacetamido)cyclohexyl)-2H-indazole-5-carboxamide Form A (5.78 kg, 12.4 mol) as a yellow solid. The XRPD of this Form A material is shown in FIG. 1.

1H NMR (500 MHZ, CDCl₃) δ ppm 1.7 (qd, J=12.7, 3.2 Hz, 2H) 1.8-2.0 (m, 2H) 2.1-2.2 (m, 2H) 2.1 (s, 3H) 2.4-2.4 (m, 2H) 2.9 (s, 3H) 4.2-4.2 (m, 3H) 4.3-4.4 (m, 1H) 4.6-4.7 (m, 1H) 6.9-7.0 (m, 1H) 7.2 (s, 1H) 7.9 (dd, J=9.1, 1.4 Hz, 1H) 8.1 (s, 1H) 8.3-8.4 (m, 1H) 8.4 (s, 1H) 8.8 (s, 1H) 11.2 (s, 1H).

### Crystallisation Studies

### Generation of Amorphous Material

Amorphous Compound (I) was generated by dissolving 3 g of Compound (I) Form A in 60 ml of trifluoroethanol/water (50:50) by stirring at 50° C. for 0.5 h, freezing the resultant solution by plunging into a liquid N₂ bath, and then lyophilising in a Freeze Dryer (Christ Alpha 2-4 LD, see www.martinchrist.de). The amorphous nature of the recovered solid was confirmed by XRPD analysis. A 91% mass recovery was obtained.

### Form A

Amorphous Compound (I) (20 mg) was suspended in 2-propanol (800 μL) and stirred at room temperature for 2 weeks. The resultant solid was collected by filtration. XRPD Analysis of the material following drying at ambient temperature revealed the solid to be the Form A anhydrous form of Compound (I) (see diffractogram of FIG. 1). Exposure of the Form A solid to accelerated aging conditions (40° C., 75% relative humidity) for 2 days and re-analysis by XRPD showed that Form A was stable under accelerated aging conditions.

Analogous experiments with ethers (1,2-dimethoxyethane, diethyl ether, THF, t-butyl methyl ether, anisole), esters (ethyl acetate, ethyl formate, isopropyl acetate), alcohols (methanol, ethanol, 1-propanol), ketones (acetone, methyl isobutyl ketone), acetonitrile and DMSO all delivered Compound (I) Form A.

The physical and chemical stability of N-(Imidazo[1,2-b]pyridazin-3-yl)-6-methoxy-2-((1r,4r)-4-(N-methylacetamido)cyclohexyl)-2H-indazole-5-carboxamide Form A was further assessed under stressed conditions (elevated temperature (70° C.) at low (11%) and high (75%) relative humidity) over a period of 21 days according to standard protocols. Briefly, samples of Form A material were weighed into sample vials, the sample vials were placed in separate jars each containing a MadgeTech temperature and humidity logger and, in a separate vial, a saturated solution of either lithium chloride (to give 11% relative humidity) or sodium chloride (to give 75% relative humidity). The two jars (low/high humidity) were then sealed and placed in a calibrated oven at a temperature of 70° C. for a period of 21 days. Control samples were maintained in sealed vials in a refrigerator for the duration of the study.

Analysis of the samples by UHPLC revealed that no organic impurities developed in the samples stored at low or high relative humidity at 70° C. over 21 days. Furthermore, XRPD confirmed that the physical form of the material over the course of the study was unchanged. It could thus be concluded that no chemical degradation of the samples or change in their physical form occurred on storage under stressed conditions. Accordingly, the suitability of N-(Imidazo[1,2-b]pyridazin-3-yl)-6-methoxy-2-((1r,4r)-4-(N-methylacetamido)cyclohexyl)-2H-indazole-5-carboxamide Form A for further development in pharmaceutical formulations was established.

### Form B

Amorphous Compound (I) (19.6 mg) was dissolved in 700 μl of THF/water/1,1,1,3,3,3-hexafluoro-2-propanol (ratio 38.6:25.7:35.7) at room temperature (RT). The resultant solution was treated with aliquots of cyclopropylmethylether (700 μL) at RT until precipitation occurred. The precipitated solid was separated from the liquid phases by centrifugation, then dried at ambient conditions (Amb.) and analyzed by HT-XRPD. Analysis reveal that this material was a trihydrate crystalline form, Form B with a XRPD as shown in FIG. 2.

### Form C

Amorphous Compound (I) (1.012 g) was dissolved in a 50/50 mixture of trifluorethanol/water (15.4 mL) then frozen (liquid N₂ bath) and dried (in a Freeze Dryer Christ Alpha 2-4 LD) overnight. The resulting amorphous material was incubated at 40° C. and 75% relative humidity for 2 days to give Compound (I) Form C.

### Compound (I) Oxalate Form

Compound (I) Form A (1 g, 1 equiv.) and oxalic acid dihydrate (273 mg, 1 equiv.) were slurried in 5 ml of ethyl acetate at ambient temperature for 4 days. Another 10 ml of ethyl acetate was then added and slurring was continued at ambient temperature for an additional 4 days at which stage the resultant solid was collected by filtration. The resultant sample of Compound (I) oxalate salt was then dried at ambient temperature prior to XRPD analysis.

### Compound (I) 3-Hydroxybenzoic Acid Form

Compound (I) Form A (1 g, 1 equiv.) and 3-hydroxybenzoic acid (299 mg, 1 equiv.) were slurried in 5 ml dichloromethane/methanol (1:1) at ambient temperature for 4 days at which stage the resultant solid was collected by filtration. The resultant sample of Compound (I) 3-hydroxybenzoic acid form was then dried at ambient temperature prior to XRPD analysis.

1. A crystalline form of N-(Imidazo[1,2-b]pyridazin-3-yl)-6-methoxy-2-((1r,4r)-4-(N-methylacetamido)cyclohexyl)-2H-indazole-5-carboxamide: 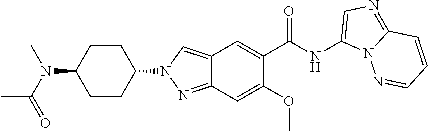 [SMILES: COc1cc2nn([C@H]3CC[C@H](N(C)C(C)=O)CC3)cc2cc1C(=O)Nc1cnc2cccnn12]
or a pharmaceutically acceptable salt or solvate thereof.

2. A crystalline form according to claim 1 that is the Form A anhydrous form characterised in that it has an X-ray powder diffraction pattern with specific peaks at about 2-theta=4.9, 12.5, 16.7, 18.8, and 23.4°.

3. A crystalline form according to claim 1 that is the Form A anhydrous form characterised in that it has an X-ray powder diffraction pattern with specific peaks at about 2-theta=4.9, 9.7, 12.5, 16.7, 18.8, 19.5, 20.7, 23.4, 25.1, and 27.4°.

4. A crystalline form according to claim 1 characterised in that it has an X-ray powder diffraction pattern substantially as shown in FIG. 1, when measured using CuKα radiation.

5. A crystalline form according to claim 1 that is the Form B trihydrate form characterised in that it has an X-ray powder diffraction pattern with specific peaks at about 2-theta==6.7, 11.3, 11.9, 17.2, and 26.1°.

6. A crystalline form according to claim 1 characterised in that it has an X-ray powder diffraction pattern substantially as shown in FIG. 2, when measured using CuKα radiation.

7. A crystalline form according to claim 1 that is a 1:1 acid addition salt of N-(Imidazo[1,2-b]pyridazin-3-yl)-6-methoxy-2-((1r,4r)-4-(N-methylacetamido)cyclohexyl)-2H-indazole-5-carboxamide and oxalic acid characterised in that it has an X-ray powder diffraction pattern with specific peaks at about 2-theta=11.2, 27.2, 3.6, 22.6 and 26.5°.

8. A crystalline form according to claim 1 characterised in that it has an X-ray powder diffraction pattern substantially as shown in FIG. 4, when measured using CuKα radiation.

9. A crystalline form according to claim 1 that is a co-crystal of N-(Imidazo[1,2-b]pyridazin-3-yl)-6-methoxy-2-((1r,4r)-4-(N-methylacetamido)cyclohexyl)-2H-indazole-5-carboxamide and 3-hydroxybenzoic acid in a 1:1 ratio that is characterised in that it has an X-ray powder diffraction pattern with specific peaks at about 2-theta=10.8, 16.5, 27.1, 18.4 and 3.4°.

10. A crystalline form according to claim 1 characterised in that it has an X-ray powder diffraction pattern substantially as shown in FIG. 5, when measured using CuKα radiation

11. A pharmaceutical formulation comprising a crystalline form according to any preceding claim and at least one pharmaceutically acceptable excipient such as a diluent or granulating agent.

12. A crystalline form according to any of claims 1 to 10 for use as a medicament or a method of treatment comprising administering a crystalline form according to any of claims 1 to 10 or a composition according to claim 11 to a patient in need thereof.

13. The use or method according to claim 12 or the pharmaceutical composition of claim 11 for use in the prevention or treatment of respiratory diseases such as asthma and chronic obstructive pulmonary disease (COPD), of cancer, of inflammatory diseases or autoinflammatory/autoimmune diseases such as systemic lupus erythematosus, rheumatoid arthritis, myositis, Sjögren's syndrome, systemic sclerosis, gout, endometriosis, atopic dermatitis and psoriasis.

14. The use or method according to claim 12 or the pharmaceutical composition of claim 11 for use in the treatment of cancer, for example a haematologic malignancy selected from Waldenstrom's macroglobulinemia (WM), non-Hodgkin lymphoma (NHL), diffuse large B-cell lymphoma (DLBCL), primary central nervous system lymphoma (PCNSL), Splenic Marginal Zone Lymphoma (SMZL), small lymphocytic lymphoma (SLL), leukaemias (chronic lymphocytic leukaemia (CLL)) and monoclonal gammopathy of undetermined significance (MGUS-IgM+).

15. A crystalline form of N-(Imidazo[1,2-b]pyridazin-3-yl)-6-methoxy-2-((1r,4r)-4-(N-methylacetamido)cyclohexyl)-2H-indazole-5-carboxamide according to any of claims 1 to 10 for use in the manufacture of a medicine.

16. A process for making N-(Imidazo[1,2-b]pyridazin-3-yl)-6-methoxy-2-((1r,4r)-4-(N-methylacetamido)cyclohexyl)-2H-indazole-5-carboxamide comprising reacting 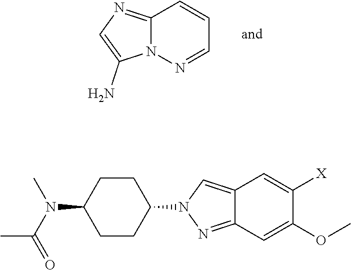
in the presence of carbon monoxide and a catalyst and wherein group X is selected from Br, Cl, I, OTf or OSO₂Me.

17. The compound N-((1r,4r)-4-(5-bromo-6-methoxy-2H-indazol-2-yl)cyclohexyl)-N-methylacetamide 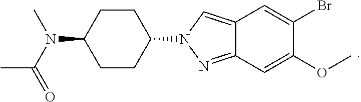 [SMILES: COc1cc2nn([C@H]3CC[C@H](N(C)C(C)=O)CC3)cc2cc1Br]
